(19) United States
(12) Patent Application Publication
(10) Pub. No.: US 2007/0092565 A1
(43) Pub. Date: Apr. 26, 2007

(54) Gastric retention drug delivery system

(75) Inventors: Jack Aurora, Lorraine (CA); Vinayak Sant, Montreal (CA)

Correspondence Address: BCF LLP;25th Floor, 1100 Rene-Levesque Boulevard West, Montreal, QC, H3B 5C9, CA

(73) Assignee: PHARMASCIENCE INC.

(21) Appl. No.: 11/257,361

(22) Filed: Oct. 25, 2005

(51) Int. Cl. A61K 9/46; A61K 9/22

(52) U.S. Cl. 424/466; 424/468

(57) ABSTRACT

A gastric retention drug delivery system (i.e. a controlled release drug dosage form) formulated so as to promote retention of the dosage form in the upper gastrointestinal tract and in particular the stomach.

The present invention relates to a gastric retention drug delivery system (i.e. a controlled release drug dosage form) which is formulated so as to promote retention of the dosage form in the upper gastrointestinal tract and in particular the stomach. Such a dosage form may be useful for many medicinal products, for example for site specific delivery in the upper gut to treat local pathology in the stomach and/or to allow a less frequent administration: i.e. once a day instead of twice a day, or twice a day instead of 3 times a day.

Gastro-retentive dosage forms are known for releasing a drug into for example at least a portion of a region defined by the stomach and the upper gastrointestinal tract. It is generally known that the location of an orally administered controlled drug delivery system in the stomach and the gastrointestinal tract as well as the rate at which a controlled drug delivery system moves from the stomach to the colon may be factors that need to be considered in the design of an oral controlled drug delivery system. It is thus known that a prolonged period of retention of the system in the stomach for example may be beneficial for various types of drugs, i.e. gastric retention systems may for example be beneficial when the drug to be administered is most effectively absorbed locally in the stomach.

One known approach that has been suggested for achieving gastric retention involves using a composition containing highly swellable polymers (i.e. swellable matrices) in admixture with a gas-generating agent to form in situ (i.e. in the stomach), a system that is large in size as well as capable of floating on gastric fluids (see for example Canadian patent application no. 2452738, the entire contents of which is incorporated herein by reference). Dosage forms containing swellable polymers in admixture with a gas-generating agent will float on gastric fluids because the gas generated and entrapped within the dosage form decreases the density of the dosage form. It is also known to manipulate the initial size and as well as the composition of an expandable drug dosage form so that it is able to swell in the stomach to a larger size, the larger size being a size which will promote the desired retention of the system in the stomach; such systems should be capable of retaining this size in the gastric fluids for sufficiently long periods under agitation conditions created by gastric motility. (see also U.S. Pat. No. 5,232,704; U.S. Pat. No. 6,120,803; U.S. Pat. No. 4,996,058; and U.S. Pat. No. 5,972,389: the entire contents of all of which are incorporated herein by reference).

It is in particular also known to use hydroxypropyl cellulose (HPC) and hydroxypropyl methylcellulose (HPMC) as components for various types of dosage forms; see for example, U.S. Pat. No. 4,871,548, U.S. Pat. No. 6,861,072, U.S. Pat. No. 6,077,538, U.S. Pat. No. 4,915,952, U.S. Pat. No. 6,090,411, U.S. Pat. No. 5,593,694 as well as U.S. Pat. No. 6,861,072 and Canadian Patent Application no. 2464322; the entire contents of all of which are incorporated herein by reference.

Notwithstanding the known gastric retention systems, such as those described in the above mentioned patent documents, it would be advantageous to have an alternative means of administering a therapeutically effective amount of a drug(s) to a patient in need thereof, in a gastric retained dosage form. Thus, the search still goes on for alternate swellable matrices comprising a gas generating component for use as part of gastro-retentive dosage forms; in particular swellable matrices comprising a gas generating component which have good swelling characteristics e.g. a good swellable matrix is one which is able to swell to up to 140% or more of its original volume within one-half hour after administration, and maintain and/or increase such swelled volume (as well as its integrity) for a desired or necessary time period thereafter (for 2 to 4 hours thereafter or longer).

### SUMMARY OF THE INVENTION

Thus, in general the present invention provides a dosage form which comprises a hydrophilic swellable matrix component which is intermingled with both a pharmaceutically active component and a pharmaceutically acceptable gas generating component (i.e. a gas component able to generate a pharmaceutically acceptable gas in the stomach).

The present invention in particular relates to dosage forms that are relatively easy to manufacture and that are able to deliver a pharmaceutically active component (e.g. one or more drugs and/or pro-drugs) in a controlled release manner to the upper gastrointestinal tract and in particular the stomach. A pro-drug is a pharmacological substance (e.g. drug) which is administered in an inactive (or significantly less active) form; once administered, the pro-drug is metabolised in the body (in vivo) into the active compound. Stated in another way, a pro-drug is an inactive precursor of a drug, converted into an active form in the body by normal metabolic processes.

The present invention more particularly relates to the realisation that a satisfactory controlled release dosage form may be formulated by exploiting a gas generating component in conjunction with a swellable matrix component provided that two conditions are met. Firstly, the swellable matrix component must be a combination of only hydroxypropyl cellulose (HPC) and hydroxypropyl methylcellulose (HPMC). Secondly, the swellable matrix must comprise specific amounts of hydroxypropyl cellulose (HPC) and of hydroxypropyl methylcellulose (HPMC) relative to each other; in particular, the matrix component weight ratio of HPC to HPMC must be such that neither the weight amount of HPC nor that of HPMC is below 20% by weight of the matrix component as a whole. Thus HPC may make up from 80 to 20% by weight of the matrix component while conversely HPMC may make up from 20 to 80% by weight of the matrix component; e.g. if HPC represents 80% by weight of the matrix component HPMC represents 20% by weight of the matrix component; if HPC represents 60% by weight of the matrix component HPMC represents 40% by weight of the matrix component; if HPC represents 40% by weight of the matrix component HPMC represents 60% by weight of the matrix component; if HPC represents 20% by weight of the matrix component HPMC represents 80% by weight of the matrix component; etc.

Accordingly, the present invention provides an oral controlled release pharmaceutical dosage form, for releasing a pharmaceutically active component (e.g. a drug) into the stomach, said dosage form comprising a combination of
a solid hydrophilic swellable matrix component, and
said pharmaceutically active component intermingled with (i.e. associated with) said matrix component,

characterized in that

said matrix component consists of a combination of hydroxypropylcellulose and hydroxypropylmethylcellulose, the weight ratio of hydroxypropylcellulose to hydroxypropymethylcellulose being from 80:20 to 20:80, (e.g. 70:30 to 30:70, preferably 60:40 to 40:60), and

said dosage form further comprises a pharmaceutically acceptable gas generating component (e.g. a carbon dioxide gas generating component) intermingled with said matrix component,

wherein the matrix component and the gas generating component are each respectively present in an amount whereby upon contact with gastric fluid said matrix component is able to swell to a larger size for promoting retention of the dosage form in the stomach and said gas generating component is able to generate sufficient gas (i.e. gas bubbles, e.g. carbon dioxide bubbles) to promote flotation of the dosage form in the stomach for promoting such retention.

The present invention in one aspect provides an oral controlled release pharmaceutical dosage form, for releasing a pharmaceutically active component (e.g. a drug) into the stomach, said dosage form consisting of a combination of
a solid hydrophilic swellable matrix component, and
said pharmaceutically active component intermingled with (i.e. associated with) said matrix component,
   characterized in that
   said matrix component consists of a combination of hydroxypropylcellulose and hydroxypropylmethylcellulose, the weight ratio of hydroxypropylcellulose to hydroxypropymethylcellulose being from 80:20 to 20:80,
   said dosage form further comprises a pharmaceutically acceptable gas generating component (e.g. a carbon dioxide gas generating component) intermingled with said matrix component,
   and optionally (i.e. as necessary or desired), said dosage form may further comprise a pharmaceutically acceptable additive component comprising one or more members selected from the group consisting of pharmaceutically acceptable lubricants, diluents, binders, disintegrants, and glidants,
   wherein the matrix component and the gas generating component are each respectively present in an amount whereby upon contact with gastric fluid said matrix component is able to swell to a larger size for promoting retention of the dosage form in the stomach and said gas generating component is able to generate sufficient gas (i.e. bubbles, e.g. carbon dioxide bubbles) to promote flotation of the dosage form in the stomach for promoting such retention.

In accordance with the present invention, the expression “consisting of” is to be understood as characterising a component or combination of components (e.g. the dosage form itself, the pharmaceutically active component, the matrix component, the pharmaceutically active component etc.) as comprising only the specified element(s) of the combination or component; but does not exclude the possible presence of minor amounts of another impurity material(s) which may have been initially associated with one or more starting materials used to formulate the dosage form (e.g. tablet) or component thereof. Thus a dosage form or a component thereof characterised by the above expression may comprise one or more materials which may be considered as pharmaceutically acceptable impurities, the impurity(ies) being of a kind and being present in an amount(s) which still provides a pharmaceutical acceptable drug form or component thereof, i.e. the presence of such other material(s) do(es) not adversally affect the function of the drug form components nor the end use of the drug form. In other words, a dosage form or a component thereof characterised by the above expression is one which conforms to acceptable drug formulation practice(s), e.g. the above expression characterizes a dosage form or component thereof as being at least substantially of the specified materials.

In accordance with the present invention the matrix component of the dosage form may comprise a solid monolithic matrix component associated with a pharmaceutically active (i.e. drug) component and a gas generating component intermingled therewith. In accordance with the present invention the dosage form may have the form of a mono-form body. Thus the dosage form may be a mono-form (i.e. a monolithic) tablet (e.g. a controlled-release oral drug dosage form) for releasing a drug into the stomach.

It is to be understood herein that a mono-form body may be a solid dosage form such as a for example a tablet made from a simple blend of components or a tablet made from a mixture of granules and non-matrix and non-drug components, the granules containing the matrix component, drug component and a gas generating component.

It is also to be understood herein that a mono-form body (i.e. tablet) as contemplated by the present invention is, a form which is monolithic in nature, i.e. of essentially uniform but not necessarily homogeneous make-up or composition. A mono-form body may thus be a body obtained by compression of a simple powder mixture comprising a matrix component, a pharmaceutically active component and a gas generating component in dry powder form or a body obtained by compression of a mixture of components wherein the mixture comprises granules as well as non-matrix and non-drug components in dry powder form, the granules having been obtained from the dry granulation, wet granulation, compaction or extrusion of a simple mixture of a matrix component, a pharmaceutically active component and a gas generating component (e.g. the mono-form body may be a tablet made-up of a single essentially uniform body (e.g. single layer)).

It is to be understood herein that a mono-form body, such as for example a (mono-form) tablet, as contemplated by the present invention is, unless otherwise indicated, a dosage form which is free or essentially free of a (known) functional or non-functional coating or layer (i.e. the dosage form is an uncoated or single layered dosage form). A functional coating may for example be one which also comprises an active pharmaceutical component (i.e. drug).

It is to be understood herein, that if a “class”, “range”, “group of substances”, etc. is mentioned with respect to a particular characteristic (e.g., temperature, weight ratio, concentration, time, (number average) molecular weight, viscosity, and the like) of the present invention, the present invention relates to and explicitly incorporates herein each and every specific member and combination of sub-classes, sub-ranges or sub-groups therein whatsoever. Thus, any specified class, range or group is to be understood as a shorthand way of referring to each and every member of a class, range or group individually as well as each and every possible sub-class, sub-range or sub-group encompassed therein; and similarly with respect to any sub-class, sub-ranges or sub-groups therein. Thus, for example, as mentioned herein
with respect to the weight ratio of HPC:HPMC, the mention of the range of 80:20 to 20:80, is to be understood herein as specifically incorporating each and every sub-range as well as each individual weight ratio of HPC:HPMC such as, for example, 70:30 to 30:70, 70:30 to 35:65, 70:30 to 45:55, 65:35 to 35:65, 60:40 to 40:60, 32:68, 45:55, 70:30, etc.;
the mention that the matrix component comprises from 40% to 98% by weight of the dosage form is to be understood herein as specifically incorporating each and every sub-range as well as each individual weight amount such as for example 45% to 80%, 50% to 75%, 40%, 49.5%, 50%, 90% etc.;
mention that the weight ratio of the pharmaceutically active component to the dosage form (e.g. tablet) may be from 0.05:99.95 to 60:40 is to be understood herein as specifically incorporating each and every sub-range as well as each individual weight ratio such as for example; in particular 1:1.24.
mention that the gas generating component which comprises at least one carbon dioxide-generating agent may be present in an amount of from 0.5 to 3% by weight of the dosage form is to be understood herein as specifically incorporating each and every sub-range as well as each individual weight;
mention that hydroxypropyl cellulose having a viscosity of from 2 centipoise (cps) to 4000 centipoise(cps) is to be understood herein as specifically incorporating each and every sub-range as well as each individual viscosity,
and similarly with respect to any other parameters whatsoever (for example, molecular weight), etc.

It is in particular to be understood herein that for any group or range, no matter how defined, a reference thereto is a shorthand way of mentioning and including herein each and every individual member described thereby as well as each and every possible class or sub-group or sub-class of members whether such class or sub-class is defined as positively including particular members, as excluding particular members or a combination thereof; for example an exclusionary definition for a formula may read as follows: “provided that when one of A and B is —X and the other is Y, —X may not be Z”.

Matrix Component

The weight ratio of hydroxypropylcellulose to hydroxypropymethylcellulose for the swellable matrix component may, as mentioned, be from 80:20 to 20:80; in other words the swellable matrix must contain a minimum amount of each of hydroxypropylcellulose and hydroxypropymethylcellulose. The weight ratio of hydroxypropylcellulose to hydroxypropymethylcellulose may, for example, be from 70:30 to 30:70, from 70:30 to 35:65, and more particularly from 60:40 to 40:60.

The matrix component itself may comprise from 40% to 98% (e.g. 49.5%) by weight of the dosage form.

The hydrophilic polymers which are suitable for forming a swellable hydrophilic polymer matrix may be chosen from:
hydroxypropyl cellulose having a viscosity of from 2 centipoise (cps) to 4000 centipoise(cps), (e.g. from 2 to 500 cps, from 150 to 400 cps, from 6 to 10 cps) measured as a 2% by weight solution in water 20° C.;
hydroxypropyl methylcellulose having a viscosity of from 2.4 centipoise (cps) to 120,000 centipoise(cps), (e.g. from 50 to 120,000 cps, from 4000 to 120,000 cps, from 80,000 to 120,000 cps, 100000 cps) measured as a 2% by weight solution in water 20° C.;
hydroxypropyl cellulose having a molecular weight (weight averaged) of from 80,000 to 10,000,000 daltons, (e.g. a molecular weight of from 80,000 to 1,150,000);
hydroxypropyl methylcellulose having a number average molecular weight (weight averaged) of 10,000 to 1,500,000;
etc.

The release of pharmaceutically active component (e.g. drug) may be facilitated by the use of a relatively low molecular weight hydroxypropylcellulose and hydroxypropylmethylcellulose provided that the minimum amounts of each of these cellulose ethers as specified herein is respected.

HPC grades of varying viscosity and degree of substitutions of hydroxypropyl groups may be used. Some representative examples are as follows:
Different grades of HPC (klucel®) are available from Herculis Incorporated USA with molecular weights of about 80,000 to about 1,150,000; Nisso HPC® types -SSL, -SL, -L, -M, -H with viscosity ranges between 2 to 4000 mPa·s (viscosity of aqueous solution containing 2% by weight of dry HPC at 20° C.) and other commercially available grades.
Hydroxy propyl cellulose—low substituted of different grades such as LH-11, LH-21, LH-31, LH-22, LH-32, LH-20, LH-30 and other available commercial grades from Shin-Etsu Chemicals Japan.

HPMC grades of varying viscosities and degree of substitution such as HPMC-2208, HPMC-2906, HPMC-2910. Some representative examples include Methocel® from Dow Chemicals USA with viscosity ranges between about 4 to 120000 mPa·s (viscosity of 2% w/v aqueous solution at 20° C.)

Gas Generating Component

A gas generating component may be intermingled with the matrix component in any manner whatsoever keeping in mind the purpose thereof (i.e. a gas generating component may, for example, be dispersed in the matrix component). As mentioned the gas generating component is present in an amount whereby upon contact with gastric fluid said gas generating component is able to generate sufficient gas (i.e. gas bubbles, e.g. carbon dioxide bubbles) to promote flotation of the dosage form in the stomach for promoting retention of the dosage form in the stomach. The function of the gas generating component is thus to form gas in situ (i.e. in the stomach) in the form of gas bubbles in the dosage form (i.e. relative to the matrix component). These gas bubbles contribute toward the expansion of the matrix component by gas inflation. These gas bubbles also contribute toward the flotation of and then maintenance of the dosage form at the surface of the liquids contained in the stomach. The floatation of the dosage form may thus increase the gastric residence time (i.e. promote residence in the stomach) of the dosage form (e.g. tablet) and result in a relatively prolonged release of the drug in the acidic environment. In addition, the floatability of the dosage form may enhance the total mean gastrointestinal residence time and allow for increased drug bioavailability.

A gas generating component may comprise at least one gas generating agent. Such agents, including mixtures of agents, may, for example, be selected from among substances capable of releasing pharmaceutically acceptable gases such as for example carbon dioxide or nitrogen; gas generating agents may for example be selected from among pharmaceutically acceptable mono- and di-basic salts of carbonic acid, ammonium carbonate and sodium azide.

A gas generating component which is suitable in a pharmaceutical composition according to the invention may for example comprise at least one carbon dioxide-generating agent. The carbon dioxide-generating agent(s) may be an alkali metal carbonate, an alkaline-earth metal carbonate, (such as calcium carbonate), or an alkali metal bicarbonate (preferably sodium bicarbonate).

In accordance with the present invention, the amount of intermingled gas generating component (i.e. intermingled with any of the other dosage form materials including any granules thereof) is to be chosen keeping in mind the purpose of the dosage form herein, namely, to provide an oral controlled release pharmaceutical dosage form, for releasing a drug into the stomach. A gas generating component may comprise, for example, from 0.5 to 3% by weight of the dosage form; the dosage form may comprise lesser or greater amounts of gas generating component depending on the amount and/or nature of the other dosage form components and may be determined empirically keeping in mind the purpose thereof. In particular a gas generating component may be one which comprises at least one carbon dioxide-generating agent which may be present in an amount of from 0.5 to 3% by weight of the dosage form.

In accordance with the present invention all of the gas generating component may be intermingled with the matrix component and the pharmaceutically active component (along with any other desired or necessary materials), i.e. for example, from 0.5 to 3% by weight (of the dosage form) of gas generating component may be intermingled with the matrix component and the pharmaceutically active component (along with any other desired or necessary materials), and the obtained blend compressed into tablets.

As an alternative, (i.e. as desired or necessary) a portion of the gas generating component may be intermingled (i.e. as an intragranule addition) with the matrix component and the pharmaceutically active component (along with any other desired or necessary intragranule materials) to form an intermediate blend; granules may be formed from the intermediate blend; and the remaining portion of the gas generating component may be intermingled (i.e. as an extragranule addition) with such granules (along with any other desired or necessary materials) to form a further blend which may then be compressed to form tablets. In accordance with this alternative approach, the amount of the portion of gas generating component used as an intragranule addition and as an extragranule addition is to be chosen keeping in mind the purpose of the dosage form herein, namely, to provide an oral controlled release pharmaceutical dosage form, for releasing a drug into the stomach. Thus, for example, a minimum of 0.1% by weight (of the dosage form) of the gas generating component may be added as an intragranule addition (i.e. while mfg. granules). In particular, the gas generating component may, for example be subdivided into 0.5 to 2.0% by weight (of the dosage form) as an intragranule addition and 1.0 to 2.5% by weight (of the dosage form) as an extragranule addition.
As a further alternative, (i.e. as desired or necessary) the matrix component and the pharmaceutically active component (along with any other desired or necessary intragranule materials) may be intermingled to form an intermediate (non-gas generating) blend; granules may be formed from the intermediate (non-gas generating) blend; and the gas generating component may be intermingled (i.e. as an extragranule addition) with such granules (along with any other desired or necessary materials) to form a further blend which may then be compressed to form tablets.
   If the active drug component is of basic nature, it may, if possible, be necessary to adjust the acidic content of the dosage form to facilitate in situ gas generation. Thus, a gas generating component may additionally comprise at least one acidic compound chosen from the group consisting of monocarboxylic acids, polycarboxylic acids as well as partial salts of polycarboxylic acids. Such acidic compounds include lactic acid, tartaric acid, maleic acid, malonic acid, malic acid, fumaric acid, succinic acid, tartaric acid, ascorbic acid, adipic acid and citric acid and partial salts thereof, such as monosodium citrate.

The gas generating component may further include other types of substances used in effervescent mixtures. The gas generating component may thus, for example, comprise sodium dihydrogen phosphate, disodium hydrogen phosphate, sodium tartrate, sodium ascorbate or sodium citrate. Yeasts which are likewise capable of generating carbon dioxide gas (e.g. baker's yeast) may also be used as a gas source; in this case the gas generating component may additionally comprise the necessary nutrients, for example glucose.

Pharmaceutically Active (e.g. Drug) Component

The weight ratio of the pharmaceutically active component to the dosage form as a whole (e.g. tablet form) may be from 0.05:99.95 (for low drug loading medicaments) to 60:40 (high drug loading medicaments; for example the weight ratio of pharmaceutically active component (i.e. drug) to tablet in particular may be from 1:1.24 to 1:1.5 (e.g. the weight ratio of drug to tablet may be 40:60).

As mentioned above, the pharmaceutically active component is intermingled with the matrix component (i.e. the pharmaceutically active component may be dispersed in the matrix component). In relation to the matrix component itself, the weight ratio of the pharmaceutically active component to the matrix component may be from 0.1:99.9 to 70:30. In particular the weight ratio of drug:matrix component may be 44.6:55.4 (this is same as the ratio of 1:1.24 mentioned above in relation to the tablet weight).

In accordance with the present invention a pharmaceutically active component (e.g. a drug) is any substance that may be used in the diagnosis of, treatment of, relief of a symptom of, or prevention of, an illness, disease or injury, including any substance that may be used to modify a chemical process or processes in the body (e.g. a mammalian body, in particular a human being's body).

The pharmaceutically active component may comprise one or more drugs and/or one or more pro-drugs with respect to which it is desired to facilitate retention in the gastrointestinal tract e.g. for drug absorption. The dosage form may, for example, be used beneficially with any drug having a significant absorption in the upper gastrointestinal tract. A gastric retained dosage form may be particularly beneficial for delivery of a drug wherein the preferred region of absorption is in the upper gastrointestinal tract (e.g. in the stomach).

The pharmaceutically active component may, for example, comprise one or more drugs selected from the group consisting of gabapentin, metformin hydrochloride, losartan potassium, sodium valproate, valproic acid, ciprofloxacin base, ciprofloxacin hydrochloride, captopril, ranitidine hydrochloride, diltiazem hydrochloride, acyclovir etc.; additional representative example drugs may be found in U.S. Pat. No. 6,261,601, the entire contents of which is incorporated herein by reference.

In particular, U.S. Pat. No. 4,087,544, for example, discloses gabapentin (1-(aminomethyl) cyclohexane acetic acid) and various analogs thereof. Gabapentin pro-drugs are also described in U.S. Pat. No. 6,683,112. The entire contents of these two U.S. patents are incorporated herein by reference.

The gastric retention delivery system of the present invention may be used for the delivery of a drug which may have anticonvulsant activity such as, for example, gabapentin, an analogue thereof, a pro-drug thereof or a pharmaceutically acceptable salt thereof. As used herein, the term “pharmaceutically acceptable salt” refers to salts that are physiologically tolerated by a user.

Gabapentin, a water soluble compound is one of the most widely used antiepileptic agents used for adjuvant therapy in the treatment of partial seizures with and without secondary generalization in adults with epilepsy. It is absorbed by an active and saturable transport system. Therefore, its oral bioavailability is not dose proportional i.e. as dose increased, bioavailability decreases. Its bioavailability decreases from 60% to 34% upon increase in the dose from 900 to 2400 mg/day given in 3 divided doses. Because of its multiple administrations per day, a missed dose can result in fluctuations in plasma levels of gabapentin, which is very critical for any antiepileptic drug. Therefore, it is a very good candidate for sustained release dosage form with once or twice a day administration.

Gabapentin has appreciable absorption in the upper gastrointestinal tract. A dosage form retainable in the stomach may thus be particularly beneficial for delivery of gabapentin, i.e. the dosage form would be able to maintain a sustained presence in the preferred region of absorption (e.g. in the stomach).

Gabapentin may be used in the free amphoteric form. Pharmaceutically acceptable salt forms that retain the biological effectiveness and properties of gabapentin and are not biologically or otherwise undesirable may also be used. As used herein, the term “gabapentin” if used alone (there being no direct or indirect indication to the contrary) is intended to include the compound itself, pro-drugs thereof as well as its pharmaceutically acceptable salts.

Pharmaceutically acceptable salts may be amphoteric and may be present in the form of internal salts. Gabapentin may form acid addition salts and salts with bases. Exemplary acids that can be used to form such salts include, by way of example and not limitation, mineral acids such as hydrochloric, hydrobromic, sulfuric or phosphoric acid or organic acids such as organic sulfonic acids and organic carboxylic acids. Salts formed with inorganic bases include, for example, the sodium, potassium, lithium, ammonium, calcium, and magnesium salts. Salts derived from organic bases include, for example, the salts of primary, secondary and tertiary amines, substituted amines including naturally-occurring substituted amines, and cyclic amines, including isopropylamine, trimethylamine, diethylamine, triethylamine, tripropylamine, ethanolamine, 2-dimethyl aminoethanol, tromethamine, lysine, arginine, histidine, caffeine, procaine, hydrabamine, choline, betaine, ethylenediamine, glucosamine, N-alkylglucamines, theobromine, purines, piperazine, piperidine, N-ethylpiperidine, fumarate, maleate, succinate, acetate and oxalate.

Herein after reference will be made to “gabapentin” by way of example only.

Ancillary Additive(s)

Pharmaceutically acceptable glidants, lubricants and other additives such as are well known to those of skill in the art, may also be included in the gastric retained dosage form, i.e. any such additive(s) may for example be included in the formulation in an amount of from 0.01% to 10% of the weight of the dosage form. For example a glidant is a substance added to the granulation in order for the granules to flow from a hopper onto a tablet press to the dies and for consistent and uniform fill. As used herein, in relation to other additives, the term “pharmaceutically acceptable” characterises the additive compounds as compounds that are compatible with the other ingredients in a pharmaceutical formulation and not injurious to the subject when administered in therapeutically effective amounts.

A dosage form of the present invention (i.e. a sustained release tablet) may for example optionally contain a pharmaceutically acceptable additive component comprising one or more members selected from the group comprising (e.g. consisting of)
a lubricant or anti-adherent (such as, for example, magnesium stearate sodium stearyl fumarate, zinc stearate, stearic acid, glyceryl behanate, glyceryl monostearate, etc.);
a glidant (such as, for example, talc, colloidal silicon dioxide, or any other silica etc);
a binder (such as, for example, polyvinylpyrrolidone (PVP), starch, gelatine, ethyl celluose sodium carboxy methyl cellulose);
a diluent (such as, for example, lactose, microcrystalline cellulose, dicalcium phosphate, sugars such as mannitol, sorbitol etc.); and
a disintegrant (such as, for example, Croscarmalose sodium, sodium starch glycolate, cross linked PVP, starch etc.).

In any event, it is to be kept in mind that any such ancillary additive(s), if present, is/are of course to be chosen and to be incorporated into the dosage form in amounts, keeping in mind the purpose of the dosage form herein, namely, to provide an oral controlled release pharmaceutical dosage form, for releasing a drug into the stomach.

Non-Functional Coating:

The dosage form (i.e. tablet) may be uncoated or may be coated with commonly used non-functional aqueous or non-aqueous coating compositions. Examples of commercially available aqueous coating formulations include Opadry®, Opadry II®, Opadry-AMB®etc. (from Colorcon USA) As a representative example, qualitative composition of Opadry II white YS-22-18096 is provided along with. Opadry II white YS-22-18096 contains titanium dioxide, polydextrose, HPMC 2910 (3cP), HPMC 2910 (6cP), HPMC 2910 (50cP), triethyl citrate and PEG 8000. A representative example of non-aqueous coating composition is as follows: HPC-L, PEG-400, talc, titanium dioxide and ethanol. Any pharmaceutically acceptable solvent can be used in the non-aqueous coating composition.

Dosage Form Preparation

The pharmaceutically active component may for example comprise 40% by weight of the dosage form.

A typical dosage form may provide for a drug delivery profile such that pharmaceutically active component may for example be delivered for at least 2 to 8 hours, and typically over a time period of about 2 to 24 hours. In order to provide for sustained delivery, the dosage form may, for example, be formulated such that at least 30 to 40 wt % of pharmaceutically active component is retained in the dosage form after 1 hour and after about 6-12 hours 40 to 100 wt % of pharmaceutically active component has been administered. The dosage form may of course be formulated for any other desired or necessary drug delivery profile.

The dosage form (e.g. tablet) of the invention may be produced in the following way: powders and/or granules are mixed together using the current production techniques

Thus as mentioned above a dosage form may be obtained by compression of a simple powder mixture comprising a matrix component and a pharmaceutically active component in dry powder form. A dosage form may as well be obtained by compression of a mixture of components wherein the mixture comprises non-matrix and non-drug components in dry powder form and granules, the granule having been obtained from the dry granulation, wet granulation, compaction or extrusion of a simple mixture of a matrix component and a pharmaceutically active component in dry powder form (e.g. the mono-form body may be a tablet made-up of a single essentially uniform body (e.g. layer)).

An example composition of a dosage form in accordance with the present invention made by wet granulation may be as shown in Table A below:

TABLE A

| Ingredient | % | Mg/tab |
|---|---|---|
| Gabapentin | 40 | 400 |
| Hydroxypropyl cellulose (LH11) | 28.22 | 282.2 |
| Hydroxypropyl methyl cellulose K100M CR | 21.28 | 212.8 |
| Povidone K-90 | 7 | 70 |
| Sodium bicarbonate | 1.5 | 15 |
| Talc | 1 | 10 |
| Magnesium stearate | 1 | 10 |
| Total | 100 | 1000 |

Povidone K-90 (PVP): as Binder; Talc: as Glidant & lubricant; Magnesium stearate: as Lubricant; LH11 from Shin-Etsu Chemicals Japan and K100M CR from Dow Chemicals USA

In the drawings which illustrate example embodiments of the present invention:

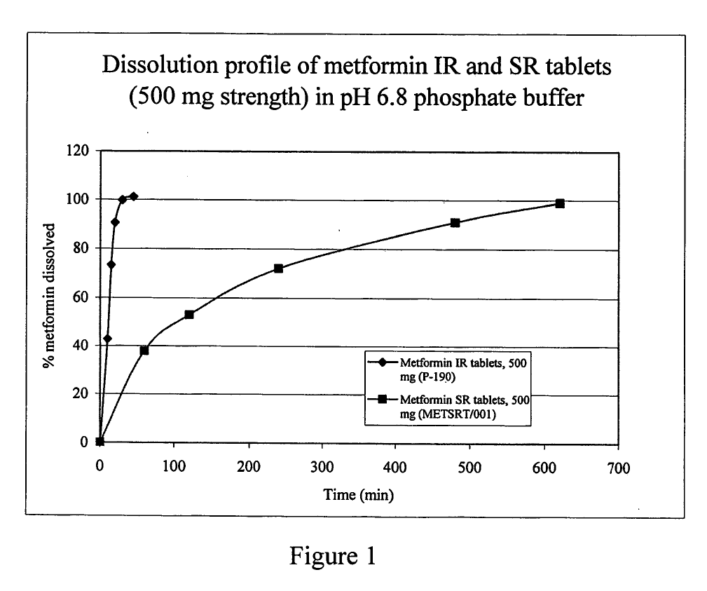

FIG. 1 is a graph illustrating the dissolution profile of meformun IR and SR tablets.

For the following Gabapentin was used in the base form and was from Zambon Group SpA. Metformin hydrochloride was from Ferico Labs. The hydroxypropylcellulose (HPC) used here in after was mfg. by Nippon Soda Co. Ltd. Based in Japan, namely L-HPC and has viscosity range of 6 to 10 mPa s./cps (2% solution at 20C). The hydroxypropylmethylcellulose used here in after was from Dow Chemicals Inc. The hydroxypropylmethylcellulose (HPMC) used in was Methocel K 100M (HPMC chemically) with a nominal viscosity of 100,000 and range of 80,000 to 120,000 mPa·s or centipoises for 2% concentration at 20C Other grades available have viscosity range of 2.4 to 120,000 mPa s.

The tablets for the trials reported below in table 1 and identified by lot numbers 049, 050, 051, 078, 079, 132 were formulated by a dry blend process, namely a direct compression. The dry blend process (direct compression process) comprised the following steps:

1. Gabapentin, HPMC, HPC and sodium bicarbonate were passed through a no. 20 mesh (US) screen and the obtained screened material was mixed in a polyethylene bag;

2. Colloidal SiO2 was mixed with part of the blend from step 1 and passed through a no. 20 mesh (US) screen. The obtained screened material was added to the blend of step 1 and the whole was mixed in the polyethylene bag;

3. Mg stearate was passed through no. 40 mesh (US) screen, and the obtained screened material was added to the blend of step 2 and mixed in the polyethylene bag to obtain a final blend ready for compression; and

4. The obtained final blend was then compressed into tablets.

The results of swelling studies in Table 1a suggest that direct compression process can be used for preparing Gabapentin SR tablets (herein the initials SR means “sustained release”).

TABLE 1
Ratio of HPC:HPMC used in different lots:

| Composition (% wgt. of tablet) | Lot 049 | Lot 050 | Lot 051 | Lot 078 | Lot 079 | Lot 132 |
|---|---|---|---|---|---|---|
| Gabapentin | 40 | 40 | 40 | 40 | 40 | 40 |
| L-HPC (Nisso) | 50.85 | 42.37 | 32.2 | 42.37 | 32.2 | — |
| M-HPC (Nisso) | — | — | — | — | — | 22.6 |
| HPMC K100 MCR | 5.65 | 14.13 | 24.3 | — | — | 33.9 |
| HPMC K15 MCR | — | — | — | 14.13 | 24.3 | — |
| Sodium bicarbonate | 1.5 | 1.5 | 1.5 | 1.5 | 1.5 | 1.5 |
| Colloidal SiO₂ | 1 | 1 | 1 | 1 | 1 | 1 |
| Mg stearate | 1 | 1 | 1 | 1 | 1 | 1 |
| HPC:HPMC ratio | 90:10 | 75:25 | 57:43 | 75:25 | 57:43 | 40:60 |
| Tablet hardness | 7-8.5 kp | 7-9 kp | 7-8.5 kp | 7-9 kp | 10 kp | 10-12 kp |

TABLE 1a

| Swelling | | | | | | | |
|---|---|---|---|---|---|---|---|
| (% vol. Increase) | | | | | | | |
| 0 | | 100% | 100% | 100% | 100% | 100% | 100% |
| 30 | min | 112% | 145% | 151% | 123% | 142% | 152% |
| 2 | h | 99% | 169% | 200% | 165% | 190% | 193% |
| 4 | h | 98.2% | 152% | 241% | 156% | 232% | 247% |

HPC from Nippon Soda, Japan; HPMC from Dow Chemicals USA

As may be seen from the above, the lot 049 wherein the weight ratio of hydroxypropylcellulose to hydroxypropylmethylcellulose is 90:10 does not provide a satisfactory swellable dosage form.

Tablets for a lot no. 155, having the formulation set forth in Table 2 below, were made by a Dry granulation process. In the Dry granulation process, a blend of gabapentin, L-HPC, HPMC K100MCR, and one third part of the sodium bicarbonate, and one half part of the talc and Mg stearate were passed through a roller compactor to obtain sheets or ribbons. The sheets or ribbons were passed through a Comil to obtain granules; Comil being the brand name of the equipment manufactured by Quadro Engineering, Canada. The granules had a particle size distribution as set forth in Table B below wherein the percentages (unless otherwise indicated) specify the percentage by weight of the granules retained on the specified U.S. sieve no.:

TABLE B

| Sieve NO. (US) | % w/w retained |
|---|---|
| 20 | 3.1% |
| 40 | 10% |
| 60 | 3.9% |
| 100 | 6.8% |
| 200 | 26.3% |
| Sieve base (undersize fines) | 49.9% |

Extragranular ingredients (i.e. the remaining part of the sodium bicarbonate, talc and Mg stearate) were then admixed with the granules in a polyethylene bag or appropriate blender and the obtained blend compressed by rotary tablet press to obtain tablets. More particularly, the Dry granulation process comprised:
step 1. Mix intragranular components viz. gabapentin, L-HPC (LH-11), HPMC K100MCR, sodium bicarbonate (0.5% by weight of the tablet), talc (0.5% by weight of the tablet) & Mg stearate (0.5% by weight of the tablet) in a polyethylene bag. Pass this mix through the rollar compactor to obtain the sheets or ribbons.
step 2. Pass the sheets obtained in above step 1 through a Comil (Quadro Engineering, Canada) to obtain the granules.
Step 3. Pass extragranular components viz, talc (0.5% by weight of the tablet) and Mg stearate (0.5% by weight of the tablet) each individually through a no. 40 mesh sieve (US standard) manually.
Step 4. to the granules obtained at step 2, add sodium bicarbonate (1% by weight of the tablet) and the talc of above step 3, and mix for about 2 min.
Step 5. Add Mg stearate of step 3 to the blend of step 4, and mix for a short time (e.g. about 30 sec.) to obtain final blend ready for compression.

The swelling characteristics for the tablets of lot 155 were determined and are set forth in Table 2a below.

TABLE 2

| Composition (% wgt. of tablet) | |
|---|---|
| Gabapentin | 40 |
| L-HPC (LH11) | 32.2 |
| HPMC K100 MCR | 24.3 |
| Sodium bicarbonate | 1.5 |
| Talc | 1 |
| Mg stearate | 1 |
| HPC: HPMC ratio | 57:43 |
| Tablet hardness | 6-8 kp |

TABLE 2a

Swelling (% vol. Increase)

| | | |
|---|---|---|
| 0 | | 100% |
| 30 | min | 158% |
| 2 | h | 188% |
| 4 | h | 215% |

The above results shown in Table 2a of swelling studies of the tablets prepared by roller compaction indicate that dry granulation process can also be used to prepare gabapentin SR tablets.

Dissolution:

A) Dissolution of gabapentin SR Tablets:

Dissolution test was carried out with Gabapentin tablets made by a wet granulation process (see below) and which had the composition as set forth in Table 2b below:

TABLE 2b
Gabapentin SR tablet

| Composition | % wgt of tablet |
|---|---|
| Gabapentin | 40 |
| HPC | 28.2 |
| HPMC | 21.3 |
| PVP | 7 |
| Sodium bicarbonate | 1.5 |
| Talc | 1 |
| Mg stearate | 1 |

Dissolution Method Details:

Apparatus—USP apparatus 2 (paddles) from Varian, USA

RPM—50

Medium—0.1N HCl

Dissolution results are reported in table 3 below which specifies the percentage by weight (w/w) of the initial amount of gabapentin released after the specified time period:

TABLE 3

| | % gabapentin released |
|---|---|
| Time (h) | Gabapentin SR tablet |
| 1 | 23 |
| 4 | 50 |
| 8 | 70 |

B) Dissolution comparison of gabapentin tablets using an alternate dissolution method:

Dissolution Method Details:

Apparatus—USP apparatus 1 (baskets) from Varian, USA

RPM—100

Medium—Deionized water

Dissolution results using alternate dissolution method are given in table 4 below which specifies the percentage by weight (w/w) of the initial amount of gabapentin released after the specified time period:

TABLE 4

| | % gabapentin released |
|---|---|
| Time (h) | Gabapentin SR tablet |
| 1 | 19 |
| 4 | 38 |
| 6.5 | 48 |
| 8 | — |

Particle Size Distribution:

Gabapentin SR tablets were made in two lots identified as lots 327 and 332, each lot having the formulation set forth in Table 2a above. Each lot was made by a wet granulation method (see below) using granules having the particle size distribution set forth in table 5 below wherein the percentages (unless otherwise indicated) specify the percentage by weight of the granules retained on the specified U.S. sieve no.:

TABLE 5

| U.S. Sieve no (& size) | 327 | 332 |
|---|---|---|
| # 20 (850 μm) | 32.3% | 23.5% |
| # 40 (425 μm) | 26.6% | 27.1% |
| # 60 (250 μm) | 12.5% | 15.2% |
| # 80 (180 μm) | 3.6% | 9.8% |
| # 100 (150 μm) | 2.0% | 3.5% |
| Sieve base (undersized fines) | 22.9% | 20.9% |

Particle size distribution may have some impact on the floating behaviour of tablets; keeping in mind the purpose of the dosage form herein, namely, to provide an oral controlled release pharmaceutical dosage form, for releasing a drug into the stomach, the desired or necessary particle size distribution may be determined on an empirical basis. In case of lot 327, 3 out of 5 tablets float immediately in 0.1N HCl, while remaining 2 tablets float in 5 min. In case of lot 332, all tablets float immediately in 0.1N HCl.

Exploitation of dosage form using alternate drug, namely, Metformin SR tablets, 500 mg:

Tablets were prepared (by wet granulation method—see below) using metformin hydrochloride as a the pharmaceutically active component. The 500 mg strength of metformin was selected to prepare sustained release (SR) tablets. The weight ratio of HPC:HPMC was maintained at 53:47 (same as used for Gabapentin SR). Also the process as well as tablet weight was kept similar for Gabapentin SR viz. 1000 mg. The composition of metformin SR tablets is as follows in Table 6:

TABLE 6

| Composition (% wgt. of tablet) | METSRT/001 |
|---|---|
| Metformin HCl | 50 |
| L-HPC (LH11) | 23.9 |
| HPMC K100 MCR | 18.1 |
| Sodium bicarbonate | 1.5 |
| Colloidal SiO₂ | 0.5 |
| Mg stearate | 1 |
| HPC: HPMC ratio | 57:43 |
| Tablet hardness | 9-12 kp |

Floating behaviour of the above metformin SR tablets was studied in 0.1N HCl. 2 out of 5 tablets started floating immediately, additional 2 tablets started floating in 5 min and all 5 tablets were floating in 10 min. This indicates that the floating behaviour is retained irrespective of the drug used (metformin or gabapentin). The dissolution of metformin SR tablets was compared with metformin IR tablets (also prepared by wet granulation process—analogous to the process referred to below), i.e. 500 mg metformin; the initials IR herein mean immediate release. The metformin IR tablets had the composition as set forth in table 7 below:

TABLE 7

| Composition (% wgt of tablet) | P-0190 |
|---|---|
| Metformin HCL | 90.9 |
| Pregelatinized starch | 1.0 |
| Croscarmalose sodium | 1.0 |
| Microcrystalline cellulose | 3.9 |
| PVP | 1.8 |
| Mg stearate | 1.0 |
| Colloidal SiO₂ | 0.4 |

The details of the dissolution method are as follows:

| Medium: | pH 6.8 phosphate buffer |
|---|---|
| Apparatus: | USP apparatus 1 (basket) |
| RPM: | 100 |

results of dissolution tests for the metformin SR tablets and the metformin IR tablets are shown below in Table 8 which specifies the percentage by weight (w/w) of the initial amount of metformin dissolved after the specified time period:

TABLE 8

| | |
|---|---|
| Metformin IR tablets, 500 mg | Metformin SR tablets, 500 mg |
| Lot no: P-0190 | Lot no: METSRT/001 |

| Time (min.) | % dissolved | Time (min) | % dissolved |
|---|---|---|---|
| 0 | 0 | 0 | 0 |
| 10 | 42.92 | 60 | 38 |
| 15 | 73.25 | 120 | 53 |
| 20 | 90.58 | 240 | 72 |
| 30 | 99.83 | 480 | 91 |
| 45 | 101.13 | 620 | 99 |

The dissolution profile of meformun IR and SR tablets is set forth in FIG. 1

The dissolution results show that combination of HPC & HPMC significantly showed the dissolution of metformin providing sustained release behaviour.

Results of Biostudy:

The bioavailability of gabapentin SR tablets, 400 mg, made by wet granulation process (see below) was evaluated in healthy human volunteers. The pharmacokinetic parameters for this Study were as follows:

| | | |
|---|---|---|
| Parameter | Mean | CV |

| | | | |
|---|---|---|---|
| Cₘₐₓ | 2859 | ng/ml | 16% |
| Tₘₐₓ | 6 | h | 24% |

| | | |
|---|---|---|
| AUC₀₋₄₈ | 42397 | 11% |
| AUC_{0-∞} | 42592 | 11% |

wherein
CV=efficient of variance
Cmax=Peak plasma concentration;
Tmax=Time required to reach peak plasma concentration;
AUC₀₋₄₈=the area under the curve in the graph of plasma drug concentration Versus Time; The 0-48 signifies the time scale in the graph i.e. 0 to 48 hours;
AUC_{0-∞}=the area under the plasma concentration versus time curve where the curve is extrapolated to the infinity time point;
Css=Steady state plasma concentration.

The above mentioned pharmacokinetic parameters were compared for immediate release (IR) Gabapentin tablets, 600 mg; gabapentin capsules, 400 mg; and gabapentin SR tablets, 400 mg; slower and sustained plasma levels were estimated.

The composition of Gabapentin 400 mg SR tablets, is as given in Table A above; the tablets were made by wet granulation process (see below).

The Gabapentin 600 mg IR tablets were made by Wet granulation process (analogous to the wet process described below). The composition for 600 mg gabapentin IR tablets (lot no. P-744) was as follows:

| Composition | % w/w |
|---|---|
| Gabapentin | 75% |
| Pregelatinized starch | 3% |
| Croscarmalose sodium | 2% |
| Microcrystalline cellulose | 13% |
| Colloidal SiO2 | 2.5% |
| PVP | 3% |
| Mg stearate | 1.5% |

The gabapentin 400 mg capsules were made by Direct blending and filling into capsules. The composition of gabapentin 400 mg capsules was as follows:

| Composition | % w/w |
|---|---|
| Gabapentin | 75.2% |
| Lactose | 16.9% |
| Corn starch | 4.9% |
| Talc | 3% |

The comparison of pharmacokinetic parameters are shown in Table 9 below:

TABLE 9

| | | Estimated | | | |
|---|---|---|---|---|---|
| | Dosing | Average | Cₘₐₓ | Tₘₐₓ | AUC_{inf} |
| Dose | interval | Cₛₛ (ng/ml) | (ng/ml) | (h) | (ng*h/ml) |
| 400 mg | 12 h | 3549 | 2569 | 6 | 42592 |
| gabapentin SR | | | | | |
| tablets | | | | | |
| 600 mg | 8 h | 5280 | 4178 | 3 | 42243 |
| gabapentin IR | | | | | |
| tablets | | | | | |
| 400 mg | 8 h | 4252 | 3190 | 3.42 | 34023 |
| gabapentin | | | | | |
| capsules | | | | | |

These results show that gabapentin SR tablets significantly increased the Tₘₐₓ of gabapentin as compared to immediate release tablets and capsule, suggesting prolonged retention of gabapentin SR tablets in stomach. Also, the AUC for sustained release tablets was found to be higher than immediate release tablets and capsules.

Tablets Prepared by Wet Granulation Process Using Isopropyl Alcohol

The process involved following steps:
1. Prepare the granulating solution of povidone K-90 in isopropyl alcohol (6.25% w/w of PVP K-90 in isopropanol).
2. In a high shear granulator (T.K. fielder from Aeromatic Fielder Ltd. UK), add gabapentin, hydroxypropyl cellulose (HPC), hydroxypropyl methyl cellulose (HPMC) and sodium bicarbonate (1% by weight of the tablet) and perform granulation using the granulating solution of step 1.
3. Dry the granules of step 2 in fluid bed dryer (from O'Hara Technologies, Canada)_followed by sizing using comil.
4. Mix the extragranular components (i.e. remaining sodium bicarbonate (0.5% by weight of the tablet) and the talc and magnesium stearate) with the dried granules of step 3 to obtain the final blend.
5. Compress the final blend of above step on a rotary tablet press (Type-Colton, from Vector Corporation USA) with the target weight of 1000 mg and target hardness of 12 kp.

Tablets prepared by above Wet process float immediately on water or 0.1N HCl. Further the combination of HPMC and HPC-L swells rapidly in 0.1N HCl increasing to 150% and 230% of its original size in 30 min and 2 hr, respectively. This may prevent escape of tablet through pyloric sphincter, which has a diameter of 12.8±7 mm.

An example composition of a dosage form in accordance with the present invention made by the above mentioned wet granulation process may be as follows:

| | % wgt. of | |
|---|---|---|
| Ingredient | tablet | Mg/tab |
| Gabapentin (base) | 40 | 400 |
| Hydroxypropyl cellulose (LH11) | 28.22 | 282.2 |
| Hydroxypropyl methyl cellulose K100M CR | 21.28 | 212.8 |
| Povidone K-90 | 7 | 70 |
| Sodium bicarbonate | 1.5 | 15 |
| Talc | 1 | 10 |
| Magnesium stearate | 1 | 10 |
| Total | 100 | 1000 |

Povidone K-90 (PVP): as Binder;Talc: as Glident & lubricant; Magnesium stearate: as Lubricant; Gabapentin (base) from Zambon Group SpA; Hydroxypropyl cellulose (LH11) from Dow Chemicals USA; Hydroxypropyl methyl cellulose K100M CR from Shin-Etsu chemicals, Japan

1. An oral controlled release pharmaceutical dosage form, for releasing a pharmaceutically active component into the stomach, said dosage form comprising a combination of
a solid hydrophilic swellable matrix component, and
said pharmaceutically active component intermingled with said matrix component,
characterized in that
said matrix component consists of a combination of hydroxypropylcellulose and hydroxypropylmethylcellulose, the weight ratio of hydroxypropylcellulose to hydroxypropymethylcellulose being from 80:20 to 20:80, and
said dosage form further comprises a pharmaceutically acceptable gas generating component intermingled with said matrix component,
wherein the matrix component and the gas generating component are each respectively present in an amount whereby upon contact with gastric fluid said matrix component is able to swell to a larger size for promoting retention of the dosage form in the stomach and said gas generating component is able to generate sufficient gas to promote flotation of the dosage form in the stomach for promoting such retention.

2-12. (canceled)

13. An oral controlled release pharmaceutical dosage form as defined in claim 1 wherein the weight ratio of hydroxypropylcellulose to hydroxypropylmethylcellulose is from 70:30 to 30:70.

14. An oral controlled release pharmaceutical dosage form as defined in claim 1 wherein said gas generating component comprises from 0.5 to 3% by weight of the dosage form.

15. An oral controlled release pharmaceutical dosage form as defined in claim 1 wherein the gas generating component is a carbon dioxide gas generating component and comprises at least one carbon dioxide-generating agent chosen from the group consisting of an alkali metal carbonate, an alkaline-earth metal carbonate and an alkali metal bicarbonate.

16. An oral controlled release pharmaceutical dosage form as defined in claim 1 wherein the gas generating component is a carbon dioxide gas generating component and comprises sodium bicarbonate.

17. An oral controlled release pharmaceutical dosage form as defined in claim 16 wherein said gas generating component comprises from 0.5 to 3% by weight of the dosage form.

18. An oral controlled release pharmaceutical dosage form as defined in claim 1 wherein said dosage form has the form of a mono form body.

19. An oral controlled release pharmaceutical dosage form as defined in claim 1 wherein the weight ratio of hydroxypropylcellulose to hydroxypropylmethylcellulose is from 70:30 to 30:70 and wherein the gas generating component is a carbon dioxide gas generating component and comprises at least one carbon dioxide-generating agent chosen from the group consisting of an alkali metal carbonate, an alkaline-earth metal carbonate and an alkali metal bicarbonate.

20. An oral controlled release pharmaceutical dosage form as defined in claim 19 wherein said gas generating component comprises from 0.5 to 3% by weight of the dosage form.

21. An oral controlled release pharmaceutical dosage form as defined in claim 19 wherein said dosage form has the form of a mono form body.

22. An oral controlled release pharmaceutical dosage form as defined in claim 1 wherein the weight ratio of hydroxypropylcellulose to hydroxypropylmethylcellulose is from 70:30 to 30:70 and wherein the gas generating component is a carbon dioxide gas generating component and comprises sodium bicarbonate.

23. An oral controlled release pharmaceutical dosage form as defined in claim 22 wherein said gas generating component comprises from 0.5 to 3% by weight of the dosage form.

24. An oral controlled release pharmaceutical dosage form as defined in claim 23 wherein said dosage form has the form of a mono form body.

25. An oral controlled release pharmaceutical dosage form as defined in claim 1 wherein said dosage form comprises a pharmaceutically acceptable additive component comprising one or more members selected from the group consisting of pharmaceutically acceptable lubricants and glidants.

26. An oral controlled release pharmaceutical dosage form as defined in claim 13 wherein said dosage form comprises a pharmaceutically acceptable additive component comprising one or more members selected from the group consisting of pharmaceutically acceptable lubricants and glidants.

27. An oral controlled release pharmaceutical dosage form as defined in claim 19 wherein said dosage form comprises a pharmaceutically acceptable additive component comprising one or more members selected from the group consisting of pharmaceutically acceptable lubricants and glidants.

28. An oral controlled release pharmaceutical dosage form as defined in claim 23 wherein said dosage form comprises a pharmaceutically acceptable additive component comprising one or more members selected from the group consisting of pharmaceutically acceptable lubricants and glidants.

29. An oral controlled release pharmaceutical dosage form as defined in claim 28 wherein said dosage form has the form of a mono form body.

30. An oral controlled release pharmaceutical dosage form as defined in claim 1 wherein said matrix component comprise from 40% to 98% by weight of said dosage form.

31. An oral controlled release pharmaceutical dosage form as defined in claim 13 wherein said matrix component comprise from 40% to 98% by weight of said dosage form.

32. An oral controlled release pharmaceutical dosage form as defined in claim 20 wherein said matrix component comprise from 40% to 98% by weight of said dosage form.

33. An oral controlled release pharmaceutical dosage form as defined in claim 23 wherein said matrix component comprise from 40% to 98% by weight of said dosage form.

34. An oral controlled release pharmaceutical dosage form as defined in claim 29 wherein said matrix component comprise from 40% to 98% by weight of said dosage form.

35. An oral controlled release pharmaceutical dosage form as defined in claim 1 wherein said pharmaceutically active component is incorporated in said dosage form at a weight ratio of pharmaceutically active component to dosage form of from 0.05:99.95 to 60:40.

36. An oral controlled release pharmaceutical dosage form as defined in claim 1 wherein said pharmaceutically active component comprises a member selected from the group consisting of gabapentin, metformin hydrochloride, losartan potassium, sodium valproate, valproic acid, ciprofloxacin base, ciprofloxacin hydrochloride, captopril, ranitidine hydrochloride, and diltiazem hydrochloride.

37. An oral controlled release pharmaceutical dosage form as defined in claim 1 wherein said pharmaceutically active component comprises gabapentin.

38. An oral controlled release pharmaceutical dosage form as defined in claim 1 wherein said gas generating component comprises at least one acidic compound selected from the group consisting of lactic acid, tartaric acid, maleic acid, malonic acid, malic acid, fumaric acid, succinic acid, tartaric acid, ascorbic acid, adipic acid and citric acid.

39. An oral controlled release pharmaceutical dosage form, for releasing a pharmaceutically active component into the stomach, said dosage form consisting essentially of a combination of
a solid hydrophilic swellable matrix component, and
said pharmaceutically active component intermingled with said matrix component,
characterized in that
said matrix component consists of a combination of hydroxypropylcellulose and hydroxypropylmethylcellulose, the weight ratio of hydroxypropylcellulose to hydroxypropymethylcellulose being from 80:20 to 20:80,
said dosage form further comprises a pharmaceutically acceptable gas generating component intermingled with said matrix component,
and optionally, said dosage form further comprises a pharmaceutically acceptable additive component comprising one or more members selected from the group consisting of pharmaceutically acceptable lubricants, diluents, binders, disintegrants, and glidants,
wherein the matrix component and the gas generating component are each respectively present in an amount whereby upon contact with gastric fluid said matrix component is able to swell to a larger size for promoting retention of the dosage form in the stomach and said gas generating component is able to generate sufficient gas to promote flotation of the dosage form in the stomach for promoting such retention.

40. An oral controlled release pharmaceutical dosage form as defined in claim 39 wherein the weight ratio of hydroxypropylcellulose to hydroxypropylmethylcellulose is from 70:30 to 30:70.

41. An oral controlled release pharmaceutical dosage form as defined in claim 39 wherein said gas generating component comprises from 0.5 to 3% by weight of the dosage form.

42. An oral controlled release pharmaceutical dosage form as defined in claim 39 wherein the gas generating component is a carbon dioxide gas generating component and comprises at least one carbon dioxide-generating agent chosen from the group consisting of an alkali metal carbonate, an alkaline-earth metal carbonate and an alkali metal bicarbonate.

43. An oral controlled release pharmaceutical dosage form as defined in claim 39 wherein the gas generating component is a carbon dioxide gas generating component and comprises sodium bicarbonate.

44. An oral controlled release pharmaceutical dosage form as defined in claim 43 wherein said gas generating component comprises from 0.5 to 3% by weight of the dosage form.

45. An oral controlled release pharmaceutical dosage form as defined in claim 39 wherein said dosage form has the form of a mono form body.

46. An oral controlled release pharmaceutical dosage form as defined in claim 39 wherein the weight ratio of hydroxypropylcellulose to hydroxypropylmethylcellulose is from 70:30 to 30:70 and wherein the gas generating component is a carbon dioxide gas generating component and comprises at least one carbon dioxide-generating agent chosen from the group consisting of an alkali metal carbonate, an alkaline-earth metal carbonate and an alkali metal bicarbonate.

47. An oral controlled release pharmaceutical dosage form as defined in claim 46 wherein said gas generating component comprises from 0.5 to 3% by weight of the dosage form.

48. An oral controlled release pharmaceutical dosage form as defined in claim 46 wherein said dosage form has the form of a mono form body.

49. An oral controlled release pharmaceutical dosage form as defined in claim 39 wherein the weight ratio of hydroxypropylcellulose to hydroxypropylmethylcellulose is from 70:30 to 30:70 and wherein the gas generating component is a carbon dioxide gas generating component and comprises sodium bicarbonate.

50. An oral controlled release pharmaceutical dosage form as defined in claim 49 wherein said gas generating component comprises from 0.5 to 3% by weight of the dosage form.

51. An oral controlled release pharmaceutical dosage form as defined in claim 50 wherein said dosage form has the form of a mono form body.

52. An oral controlled release pharmaceutical dosage form as defined in claim 39 wherein said dosage form comprises a pharmaceutically acceptable additive component comprising one or more members selected from the group consisting of pharmaceutically acceptable lubricants and glidants.

53. An oral controlled release pharmaceutical dosage form as defined in claim 40 wherein said dosage form comprises a pharmaceutically acceptable additive component comprising one or more members selected from the group consisting of pharmaceutically acceptable lubricants and glidants.

54. An oral controlled release pharmaceutical dosage form as defined in claim 46 wherein said dosage form comprises a pharmaceutically acceptable additive component comprising one or more members selected from the group consisting of pharmaceutically acceptable lubricants and glidants.

55. An oral controlled release pharmaceutical dosage form as defined in claim 50 wherein said dosage form comprises a pharmaceutically acceptable additive component comprising one or more members selected from the group consisting of pharmaceutically acceptable lubricants and glidants.

56. An oral controlled release pharmaceutical dosage form as defined in claim 55 wherein said dosage form has the form of a mono form body.

57. An oral controlled release pharmaceutical dosage form as defined in claim 39 wherein said matrix component comprise from 40% to 98% by weight of said dosage form.

58. An oral controlled release pharmaceutical dosage form as defined in claim 40 wherein said matrix component comprise from 40% to 98% by weight of said dosage form.

59. An oral controlled release pharmaceutical dosage form as defined in claim 47 wherein said matrix component comprise from 40% to 98% by weight of said dosage form.

60. An oral controlled release pharmaceutical dosage form as defined in claim 50 wherein said matrix component comprise from 40% to 98% by weight of said dosage form.

61. An oral controlled release pharmaceutical dosage form as defined in claim 56 wherein said matrix component comprise from 40% to 98% by weight of said dosage form.

62. An oral controlled release pharmaceutical dosage form as defined in claim 39 wherein said pharmaceutically active component is incorporated in said dosage form at a weight ratio of pharmaceutically active component to dosage form of from 0.05:99.95 to 60:40.

63. An oral controlled release pharmaceutical dosage form as defined in claim 39 wherein said pharmaceutically active component comprises a member selected from the group consisting of gabapentin, metformin hydrochloride, losartan potassium, sodium valproate, valproic acid, ciprofloxacin base, ciprofloxacin hydrochloride, captopril, ranitidine hydrochloride, and diltiazem hydrochloride.

64. An oral controlled release pharmaceutical dosage form as defined in claim 39 wherein said pharmaceutically active component comprises gabapentin.

65. An oral controlled release pharmaceutical dosage form as defined in claim 39 wherein said gas generating component comprises at least one acidic compound selected from the group consisting of lactic acid, tartaric acid, maleic acid, malonic acid, malic acid, fumaric acid, succinic acid, tartaric acid, ascorbic acid, adipic acid and citric acid.
